(19) United States
(12) Patent Application Publication
(10) Pub. No.: US 2008/0127978 A1
(43) Pub. Date: Jun. 5, 2008

(54) Pressure support system with dry electrode sleep staging device

(76) Inventors: Benjamin Rubin, Boston, MA (US); Paolo DePetrillo, Boston, MA (US)

Correspondence Address: ROPES & GRAY LLP, PATENT DOCKETING 39/41, ONE INTERNATIONAL PLACE, BOSTON, MA, 02110-2624, US

(21) Appl. No.: 12/001,051

(22) Filed: Dec. 5, 2007

Related U.S. Application Data
(60) Provisional application No. 60/872,920, filed on Dec. 5, 2006.

(51) Int. Cl. A61M 16/00

(52) U.S. Cl. 128/204.23

(57) ABSTRACT

This invention relates to systems and methods for treating sleep apnea, which include a first dry electrode for detecting EEG signals of a user, positioned at or near a head of a user; a sleep stage processor for determining a sleep stage of the user based, at least in part, on the EEG signals detected by the first dry electrode, and a pressure delivery device for delivering a controllable stream of air to at least one of a nose and a mouth of the user, the stream of air having a pressure selected based, at least in part, on the sleep stage determined by the sleep stage processor.

### CROSS-REFERENCE TO RELATED APPLICATIONS

This application claims the benefit of U.S. Provisional Application No. 60/872,920 filed Dec. 5, 2006, which is hereby incorporated by reference herein in its entirety.

### BACKGROUND

Pressure support devices are used as a therapeutic treatment to correct for sleep-related breathing disorders, such as obstructive sleep apnea, central sleep apnea, and Cheyne-Stokes respiration. By delivering pressure to an airway of a user to keep the airway open, pressure support devices prevent apnea events, which may include obstructions of the airway or reductions in airflow within the airway (i.e., complete or partial obstructions). Pressure support devices deliver a constant or variable pressure, either of which may be determined based on respiratory variables or other signals from the user.

For a constant pressure support device, for example a Continuous Positive Airway Pressure (“CPAP”) device, the pressure is often determined based on polysomnography (“PSG”) signals, such as electroencephalogram (“EEG”) signals, electrooculogram (“EOG”) signals, electromyogram (“EMG”) signals, electrokardiogram (“EKG”) signals, oxygen saturation, and/or nasal or oral air flow, which are monitored and assessed by a trained clinician who adjusts the pressure during one or more all-night sleep studies to ensure the prevention of obstructions. The PSG signals are used to determine sleep stage of the user. Pressure for a CPAP may also be set by a regression model that takes into account anthropometric characteristics, neck circumference, and the frequency of nocturnal breathing abnormalities.

Variable pressure support devices include Bi-Level Positive Airway Pressure (“BiPAP”) devices, which deliver different pressures for inhalation and exhalation to increase comfort and efficacy, and Automatic Positive Airway Pressure (“APAP”) devices which automatically adjust the pressure delivered based on a record of respiratory variables detected from the user. An APAP device can estimate the pressure to deliver without an all-night sleep study and can adjust the pressure relative to changes in respiratory variables detected during a single night and/or between nights. U.S. Pat. No. 6,425,861 describes another method for providing variable pressure in which an expert operator in a central location monitors PSG signals to manually adjust a CPAP device. This method requires both a PSG system incorporating wet electrodes, which are difficult to apply and uncomfortable to wear, and an expert operator to perform adjustments.

In a home setting, however, a system for monitoring sleep stage is difficult to implement. For example, the application of a PSG system is infeasible in a home setting. Wet electrode-based EEG systems are time-consuming and messy because they usually require applying a gel or paste to act as a conductive path and abrading the skin at the point of contact to remove the outer layer of dead skin to ensure signal quality. In addition, long-term application of wet electrodes is infeasible because of the long-term effects on the skin at the point of contact. As such, in home settings, pressure support devices incorporating a sleep stage system are currently infeasible. In addition, current pressure support devices, by not being able to monitor many relevant signals from the user, are incapable of providing feedback based on such signals to the user. Current pressure support devices may also provide inappropriate pressures at different time (e.g., too much pressure may prevent a user from falling asleep or awaken a sleeping user), owing to their inability to adjust to changes in a user's condition. Because of device discomfort and the lack of feedback, users often fail to comply with a treatment regimen.

As such, a need remains for comfortable, easy-to-use pressure support devices capable of adjusting the pressure delivered to effectively treat sleep apnea in a home setting. A need also remains for pressure support devices that can provide feedback to a user to encourage user compliance with a treatment regimen.

### SUMMARY

The systems and methods described herein relates to systems and methods for treating sleep apnea, which include a first dry electrode for detecting EEG signals of a user, positioned at or near a head of a user; a sleep stage processor for determining a sleep stage of the user based, at least in part, on the EEG signals detected by the first dry electrode, and a pressure delivery device for delivering a controllable stream of air to at least one of a nose and a mouth of the user, the stream of air having a pressure selected based, at least in part, on the sleep stage determined by the sleep stage processor. In some embodiments, a pressure processor determines the pressure of the controllable stream of air based, at least in part, on the sleep stage determined by the sleep stage processor. The sleep stage may be at least one of light sleep, deep sleep, awake, asleep, REM sleep, non-REM sleep, stage 1, stage 2, stage 3, and stage 4. In some embodiments, a headband is attached to and positions the first dry electrode on the user, where the headband is adapted to encircle a head of the user. The sleep stage processor may apply a neural network when processing the EEG signals to determine the sleep stage of the user.

The pressure delivery device may include a mask positioned at or near at least one of the nose and the mouth of the user, a tube connected to the mask for delivering air to the mask, and a pump connected to the tube for generating the controllable stream of air. The pressure delivery device may include at least one of a continuous positive airway pressure device, a bilevel positive airway pressure device, and an automatic positive airway pressure device. In some embodiments, the pressure delivery device delivers a stream of air having a lower pressure when the sleep stage indicates that the user is awake than when the sleep stage indicates that the user is asleep. In some embodiments, the pressure delivery device delivers a stream of air having a lower pressure when the sleep stage indicates that the user is in REM sleep than when the sleep stage indicates that the user is in non-REM sleep. In some embodiments, the pressure delivery device delivers a stream of air having a lower pressure when the sleep stage indicates that the user is in light sleep than when the sleep stage indicates that the user is in deep sleep.

In some embodiments, a wake-up device determines a wake-up time for the user based at least partially on the sleep stage of the user. The wake-up device may select the wake-up time according to a wake-up condition received from the user and to wake the user when the sleep stage of the user is transitioning between REM sleep and non-REM sleep.

A transmitter, in communication with the first dry electrode, may wirelessly transmit the EEG signals and a receiver may wirelessly receive the EEG signals from the transmitter and transmit the EEG signals to the sleep stage processor. In some embodiments, a second dry electrode, positioned at or near the head of the user, may detect the EEG signals of the user. An electrode processor may receive and process the EEG signals from the first and second dry electrodes. In particular, the electrode processor may generate a difference between an output of the first dry electrode and an output of the second dry electrode. In addition, a third dry electrode, positioned at or near the head of the user and in communication with the electrode processor, may detect the EEG signals of the user, where the third dry electrode serves as an electrical ground. A memory, in communication with at least one of the sleep stage processor and the pressure delivery device, may store at least one of a history of sleep stages of the user and a history of pressures at which the controlled stream of air is delivered to the user.

In some embodiments, a housing contains the sleep stage processor and a display, seated on the housing, depicts information based at least partially on the sleep stage. The display may depict at least one of an indicator denoting the sleep stage of the user and a respiratory event number representing an apnea-hypopnea index. The display may also or alternatively depict at least one of the EEG signals, a hypnogram corresponding to a history of sleep stages of the user, a sleep quality index representing sleep quality of the user over a period of time, and a total sleep number representing a total amount of sleep over a period of time.

In some embodiments, an actigraph may detect motion signals representing movement by the user, where at least one of the sleep stage of the user and the pressure of the controllable stream of air is determined based, at least in part, on the motion signals.

The first dry electrode may include a conductive fabric disposed in contact with skin of the user. A portion of the first dry electrode in contact with skin of the user may be flexible. The first dry electrode may detect at least one of a level of muscle tone of the user, an EOG signal, and a galvanic skin response, where the sleep stage processor determines the sleep stage based at least partially on at least one of the level of muscle tone, the EOG signal, and the galvanic skin response

### BRIEF DESCRIPTION

The foregoing and other objects and advantages of the invention will be appreciated more fully from the following further description thereof, with reference to the accompanying drawings wherein:

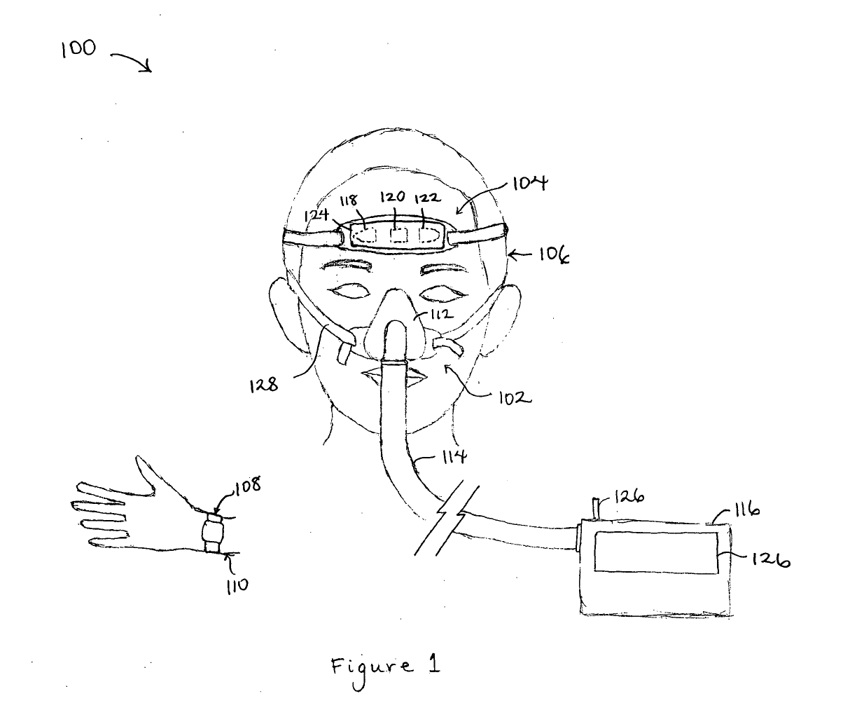

FIG. 1 depicts a pressure support system according to an illustrative embodiment of the invention;

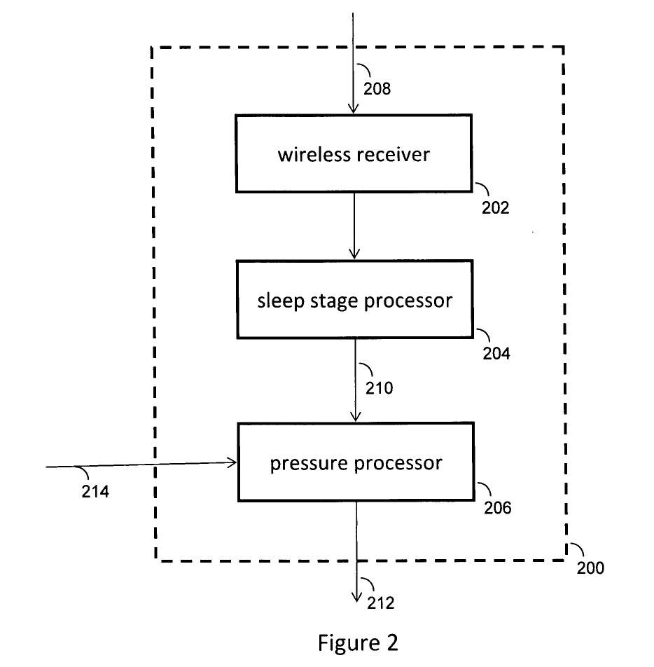

FIG. 2 depicts an exemplary block diagram that may be implemented by components within the base, according to an illustrative embodiment of the invention;

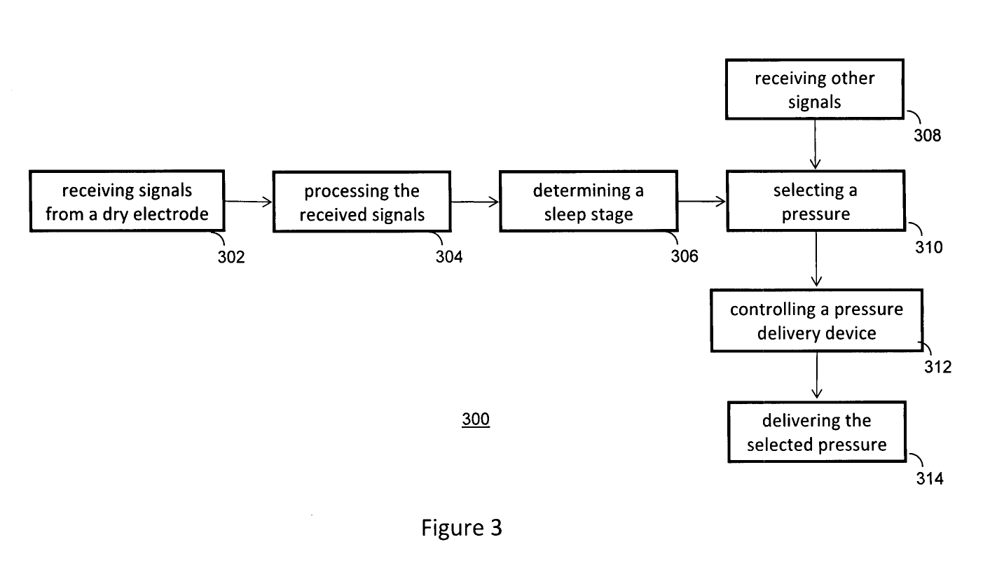

FIG. 3 depicts a flow diagram for an illustrative operation of a pressure support system, such as the pressure support system of FIG. 1; and

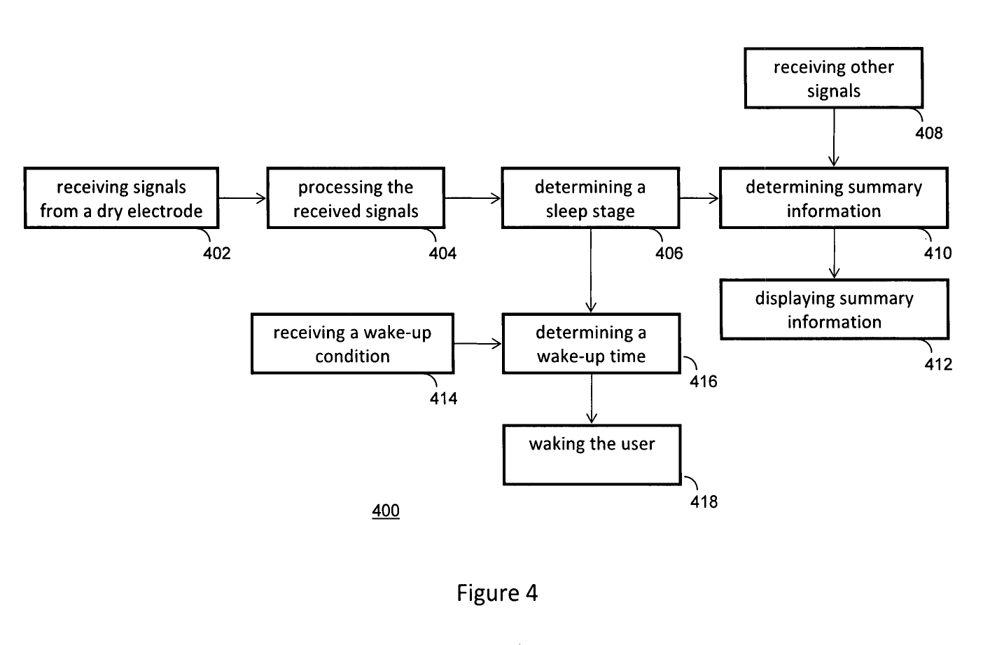

FIG. 4 depicts a flow diagram for an illustrative operation of a pressure support system, such as the pressure support system of FIG. 1.

### DETAILED DESCRIPTION

The systems and methods described herein pertains to systems and methods for treating sleep apnea in which dry electrodes detect physiological signals to determine sleep stage and information related to sleep stage and pressure in a pressure support system may be selected or adjusted based on the sleep stage and/or sleep stage related information. These physiological signals could be EEG, EMG, EKG, EOG, electrodermal activity (“EDA”), oxygen saturation, movement, and/or any other signals detectable by electrodes. Dry electrodes, especially those which are lightweight and/or flexible, are more comfortable, even over longer periods of time, than wet electrodes. They are easier to use because they may easily be applied, for example, via a headband that can be slipped on the head and placed in contact with the forehead. The dry electrodes may therefore be used in a setting that does not require a medical professional to apply the electrodes. For example, a user can apply the dry electrodes in a home setting on a regular basis. The dry electrodes can be used in conjunction with a pressure delivery device.

FIG. 1 depicts a pressure support system 100 according to an illustrative embodiment of the invention. The pressure support system 100 includes a pressure delivery device 102, a headband 104 attached to a human head 106 of a user, and an actigraph device 108 attached to a human wrist 110 of the user. The headband 104 has three dry electrodes 118, 120, and 122 (shown by outline in FIG. 1) and an electrode processor 124. The pressure delivery device 102 is capable of delivering pressure to the nostrils through a mask 112, placed over the nose of the human head 106, through a tube 114 connected to a pump contained within a base 116. The mask 112 may alternatively, or in addition, be placed over the mouth of the user. The mask 112 is held in place on the head 106 when in use by a support band 128 that surrounds the head 106. Other support structures may be used to hold the mask 112 in place. For example, the support structure holding the mask 112 in place and the headband 104 may form a unitary structure. Such a unitary structure may surround the head 106 with one or more bands. Other structures that may be worn or applied to the head 106 of the user for delivering pressure to the user are well-known in the art, such as those used in conjunction with CPAP, BiPAP, or APAP devices. Any of these such structures may be used in conjunction with the headband 104 or modified to include the three dry electrodes 118, 120, and 122 and the electrode processor 124.

The electrodes 118, 120, and 122 are disposed on an interior surface of the headband 104 such that the electrodes 118, 120, and 122 may contact the skin on the forehead of the human head 106, when the headband is worn by the user. Dry electrodes may alternatively or in addition be placed in contact with skin elsewhere on the user's body to detect physiological signals of the user.

The electrodes 118, 120, and 122 may be flexible. For example, the electrodes 118, 120, and 122 may be made of a conductive fabric, such as a silverized fabric. Other metals may also be used to render fabric conductive, such as copper, stainless steel, gold, or a blend of copper and silver. Other dry electrodes, such as capacitive electrodes, metal disk electrodes, conductive foam, conductive rubber, and micromachined spikes, may also be used. Exemplary metal disks used in electrodes may be made of stainless steel, copper, or other metals. Exemplary foam may be silverized or otherwise made conductive, and similar to conductive fabric has the advantage of being soft and pliable. Exemplary dry rubber electrodes comprise a flexible or inflexible rubber impregnated with a conductive material such as metal or carbon nanotubes. Micromachined spikes may be made of silicon, metal, or organic materials and have the advantage of being able to penetrate the layer of skin that impedes signal transmission. Exemplary dry electrodes that are capacitive as opposed to ohmic, exemplary conductive foam, and exemplary metal disk electrodes are described in “Dry and Capacitive Electrodes for Long-Term ECG-Monitoring” by Anna Karilainen, Stefan Hansen, and Jörg Müller, SAFE2005, 8th Annual Workshop on Semiconductor Advances for Future Electronics, 17-18 November 2005, Veldhoven, The Netherlands, p. 155-161. Exemplary capacitive electrodes that do not require contact with the user's skin are described in “Remote detection of human electroencephalograms using ultrahigh input impedance electric potential sensors,” by C. J. Harland, T. D. Clark, and R. J. Prance, Applied Physics Letters, Vol. 81, No. 17, Oct. 21, 2002, p. 3284-3286. Exemplary micromachined spikes are described in “Characterization of Micromachined Spiked Biopotential Electrodes” by Patrick Griss, Heli K. Tolvanen-Laakso, Pekka Meriläinen, and Göran Stemme, IEEE Transactions on Biomedical Engineering, Vol. 49, No. 6, June 2002, p. 597-604. The above references are hereby incorporated by reference herein.

The electrode processor 124 electrically connects to the electrodes 118, 120, and 122 such that electrode 120 serves as a ground. The electrode processor 124 may amplify and condition the difference between electrodes 118 and 122 to derive signals, such as EEG, EOG, EMG, EDA, and GSR signals. Alternatively, the electrode processor 124 may transmit said signals to the base 116 of the pressure delivery device 102 for processing. In some embodiments, the electrode processor 124 may include a wireless transmitter which wirelessly transmits signals for receipt by a wireless receiver disposed within the base 116. Alternatively, the electrode processor 124 may be in communication with the base 116 via a wire. For example, the wire may be integrated with the tube 114 and/or a support structure for holding the mask 112 and/or electrodes 118, 120, and 122 in place on the user. In some embodiments, the headband 104 may have two dry electrodes instead of three, such that the electrode processor 124 generates a difference between the two dry electrodes.

The headband 104 may be, for example, any of the illustrative headbands, or support structures for holding electrodes in place, described in the U.S. Application Ser. No. 11/586,196 filed Oct. 24, 2006, which is incorporated by reference herein in its entirety.

The base 116 includes a wireless antenna 126 to receive signals from the electrode processor 124 and components, such as receivers and processors implementing software, for processing the received signals. FIG. 2 depicts an exemplary block diagram 200 that may be implemented by components within the base 116, according to an illustrative embodiment of the invention. In particular, FIG. 2 depicts a wireless receiver 202 for receiving signals 208 from the wireless antenna 126, a sleep stage processor 204 for processing signals received by the wireless receiver 202, and a pressure processor 206 for processing an output 210 from the sleep stage processor 204. In particular, the sleep stage processor 204 may be a microprocessor having software capable of analyzing the signals in the frequency and time domains and implementing a neural network trained to classify sleep stages, thereby providing an output 210 indicative of a sleep stage of the user. For example, the output 210 may indicate that the user's sleep stage is asleep, awake, light sleep (i.e., stage 1 or stage 2 sleep), deep sleep (i.e., stage 3 or stage 4 sleep), REM sleep, non-REM sleep, or a sleep stage of a particular number (e.g., stage 1, stage 2, stage 3, or stage 4). The output 210 may be used by a software program on the pressure processor 206 to select a pressure 212 to deliver to the user through the tube 114 and mask 112. The pressure processor 206 may also receive other signals 214 relating to the user, which also may be used by the software program to select a pressure 212. For example, the signals 214 may originate from an actigraph 108 that measures motion events of the wrist 110 of the user and wirelessly transmit information relating to the measured motion events to the wireless antenna 126 of the base 116. In addition or alternatively, the signals 214 include respiratory variables related to respiratory events, such as a frequency of apnea events and/or hypopnea events. In some embodiments, processors and/or other components of the base 116 are disposed on the headband 104 or on another support structure capable of attaching to the head 106 of the user, instead of in a separate housing as shown in FIG. 1. For example, the sleep stage processor 204 and/or the pressure processor 206 may be disposed on the headband 104, in which case the output of the sleep stage processor and/or the pressure processor 206, respectively, may be transmitted to the base 116 for receipt by a receiver. The base 116 includes a display 126 for showing the user information relating to pressure delivered and sleep stage throughout the night and/or over multiple nights. For example, this information could include an indicator denoting the sleep stage of the user, a hypnogram corresponding to a history of sleep stages of the user, a sleep quality index representing sleep quality of the user over a period of time, a total sleep number representing a total amount of sleep over a period of time, the time spent in each stage of sleep, and/or a respiratory event number representing a sleep apnea severity over a period of time. Exemplary respiratory event numbers include a number of apnea events or complete obstructions, a number of hypopnea events or partial obstructions, an apnea index representing a frequency of apnea events, a hypopnea index representing a frequency of hypopnea events, and an apnea-hypopnea index (“AHI”) representing a frequency of respiratory events. The base 116 and/or the processors within the base 116 may have further features and capabilities such as those described in the patent applications: U.S. application Ser. No. 11/586,196 filed Oct. 24, 2006, which is incorporated by reference herein in its entirety.

FIG. 3 depicts a flow diagram 300 for an illustrative operation of a pressure support system, such as the pressure support system 100 of FIG. 1. Signals from dry electrodes, which are affixed to the user to detect physiological signals, are received at step 302 and processed at step 304. At step 306, a sleep stage is determined based on the processed signals of step 304. At step 310, a pressure is selected based on the sleep stage determined at step 306. In some embodiments, the pressure is also selected based on other signals received at step 308, either from the dry electrodes or other devices. At step 312, a pressure delivery device is controlled based on the pressure selected at step 310. At step 314, the pressure delivery device delivers the pressure selected at step 310 to the user.

The dry electrodes of step 302 may be like any of those described above with respect to FIG. 1 (e.g., electrodes 118, 120, and 122 of FIG. 1) and may detect physiological signals of the user, such as EEG, EOG, EMG, and/or GSR signals. Other signals may also be captured from the forehead and/or other locations on a body. The signals received from the dry electrodes at step 302 are processed at step 304 using a processor, such as the electrode processor 124 of FIG. 1. In some embodiments, processing the received signals includes using analog and digital techniques to amplify the difference between the signals received at step 302, filter the signals, and/or detect artifacts to be rejected. In some embodiments, signals received and processed at steps 302 and 304 are solely EEG signals.

At step 306, a sleep stage of the user may be determined by a processor, such as the sleep stage processor 204 of FIG. 2 based on the processed signals from step 304. The processor may implement a trained neural network to determine sleep stage. Sleep stage information may be inputted into a pressure determination algorithm that may be implemented by a pressure processor, such as the pressure processor 206 of FIG. 2, to select a pressure at step 310. For example, a lower pressure may be selected if the sleep stage indicates that the user is awake as opposed to sleeping. In particular, when the user is awake, the pressure selected may be negligible or zero. The selected pressure may also vary between different sleeping stages, such as between light sleep and deep sleep or between REM sleep and non-REM sleep. In particular, the pressure selected may be based on upper airway closing pressure (“UACP”), which has been shown to vary according to sleep stage, where higher pressure may be appropriate for higher UACP. For example, it has been shown that UACP is lowest during REM sleep, slightly higher in non-REM stage 1 or 2 (i.e., light sleep), and highest in non-REM stage 3 or 4 (i.e. deep sleep) (see Issa F G, Sullivan C E. Upper airway dosing pressures in snorers. J Appl Physiol. 1984; 57:528-35, which is incorporated by reference herein in its entirety). In addition, the pressure selected may be based on how much pressure a user may tolerate without waking, which may vary according to sleep stage as well. In particular, a user in a deeper sleep stage may tolerate more pressure before being aroused by discomfort from the pressure, than if the user had been in a lighter sleep stage. For example, a higher pressure may be selected for stage 3 or 4 than for stage 2, for stage 2 than for stage 1, and/or for tonic REM sleep than for phasic REM sleep. Additionally or alternatively, the sleep stage determined at step 306 may be a more precise representation of the user's sleep depth, determined by using more continuous measures of power in the EEG to gain more detailed information about sleep depth, which may in turn allow for more precise selection of the pressure at step 310. In some embodiments, the pressure determination algorithm selects a pressure based on other inputs as well. In particular, the pressure may be selected based on physiological signals detected from the user that are received at step 308, such as respiratory variables determined from monitoring airflow (e.g., the number or frequency of apnea and/or hypopnea events), other signals detectable by the dry electrodes, and/or signals from other devices applied to the user, such as an actigraph (e.g. actigraph 108 of FIG. 1).

A pressure delivery device for delivering a pressurized stream of air to the user may be controlled at step 312. Exemplary pressure delivery devices may include a pump in connection with a mask that can be worn by the user, as described above with respect to FIG. 1, a CPAP device, a BiPAP device, and an APAP device. At step 314, the pressure delivery device delivers a stream of air having the selected pressure from step 310. Alternatively, the signals detected by the dry electrodes could be used independently or in conjunction with each other to automatically adjust pressure, without step 306 (i.e., without determining the sleep stage of the user).

FIG. 4 depicts a flow diagram 400 for an illustrative operation of a pressure support system, such as the pressure support system 100 of FIG. 1 and capable of determining an optimal wake-up point and presenting sleep and respiratory summary information to a user. Steps 402, 404, 406, and 408 may be similar to steps 302, 304, 306, and 308 of FIG. 3. The process represented by flow diagram 400, or portions thereof (e.g., steps 410 and 412 or steps 414, 416, and 418) may be implemented by the same system as the process represented by flow diagram 300 of FIG. 3, or portions thereof. Signals from dry electrodes, which are affixed to the user to detect physiological signals, are received at step 402 and processed at step 404. At step 406, a sleep stage is determined based on the processed signals of step 404. At step 410, summary information, which may be related to sleep and/or respiratory events, is determined based on the sleep stage determined at step 406. The summary information may be determined based on other signals received at step 408, either from the dry electrodes or other devices. At step 412, summary information determined at step 410 is displayed to the user, for example, by a display such as display 126 of FIG. 1. In some embodiments, the user is awoken (step 418) at a wake-up time determined (step 416) based on the sleep stage determined at step 406 and a wake-up condition received at step 414.

The summary information may be determined at step 410 by a processor that uses as inputs respiratory events, sleep stage, and/or other received signals to provide summary information 412, such as an indicator denoting the sleep stage of the user, a respiratory event number representing a number or frequency of apnea and/or hypopnea events (e.g., an apnea-hypopnea index), an EEG signal, a hypnogram corresponding to a history of sleep stages of the user, a sleep quality index representing sleep quality of the user over a period of time, a total sleep number representing a total amount of sleep over a period of time, a number of arousals, a sleep depth representing proportion of time spent in deeper sleep stages over a period of time, and a time spent in a particular sleep stage over a period of time. Displaying summary information may communicate results or progress of, and/or encourage compliance with, the therapeutic treatment being implemented by the pressure support system. The information may be displayed in a home or clinical setting to a user, doctor, or other trained professional, who may modify the therapeutic treatment based on the displayed information.

The wake-up condition received at step 414 may be a latest wake-up time received from the user. The sleep stage determined at step 406 may be used to determine a wake-up time, which is near, at, or before the latest wake-up time, at which a user may prefer to be awoken or may minimize sleep inertia after being awoken. For example, the wake-up time may be determined such as to wake the user when the sleep stage of the user is transitioning between REM sleep and non-REM sleep. The wake-up time may, alternatively or in addition, be determined such as to wake the user when the sleep stage is not deep sleep. Waking the user at step 418 may include sounding an alarm, which may be auditory, visual, tactile, electric, or any other form capable of waking a sleeping user.

The above described embodiments are presented for purposes of illustration and not of limitation, and the present invention is limited only by the claims which follow. Furthermore, all of the flow diagrams and processes described above are illustrative. Steps may be added or removed to any of the flow charts, and steps may be performed in a different order.

What is claimed is:

1. A system for treating sleep apnea, comprising
a first dry electrode for detecting EEG signals of a user, positioned at or near a head of a user,
a sleep stage processor for determining a sleep stage of the user based, at least in part, on the EEG signals detected by the first dry electrode, and
a pressure delivery device for delivering a controllable stream of air to at least one of a nose and a mouth of the user, the stream of air having a pressure selected based, at least in part, on the sleep stage determined by the sleep stage processor.

2. The system of claim 1, comprising a pressure processor for determining the pressure of the controllable stream of air based, at least in part, on the sleep stage determined by the sleep stage processor.

3. The system of claim 1, wherein the pressure delivery device comprises
a mask positioned at or near at least one of the nose and the mouth of the user,
a tube connected to the mask for delivering air to the mask, and
a pump connected to the tube for generating the controllable stream of air.

4. The system of claim 1, wherein the pressure delivery device comprises at least one of a continuous positive airway pressure device, a bilevel positive airway pressure device, and an automatic positive airway pressure device.

5. The system of claim 1, wherein the sleep stage is at least one of light sleep, deep sleep, awake, asleep, REM sleep, non-REM sleep, stage 1, stage 2, stage 3, and stage 4.

6. The system of claim 1, wherein the pressure delivery device delivers a stream of air having a lower pressure when the sleep stage indicates that the user is awake than when the sleep stage indicates that the user is asleep.

7. The system of claim 6, wherein the pressure delivery device delivers no pressure when the sleep stage indicates that the user is awake than when the sleep stage indicates that the user is asleep.

8. The system of claim 1, comprising a wake-up device for determining a wake-up time for the user based at least partially on the sleep stage of the user.

9. The system of claim 8, wherein the wake-up device selects the wake-up time according to a wake-up condition received from the user and to wake the user when the sleep stage of the user is transitioning between REM sleep and non-REM sleep.

10. The system of claim 1, comprising a headband adapted to encircle a head of the user, attached to the first dry electrode, and for positioning the first dry electrode on the user.

11. The system of claim 1, comprising a support structure through which the controllable stream of air is delivered and attached to the first dry electrode, for positioning the first dry electrode on the user.

12. The system of claim 1, comprising
a transmitter in communication with the first dry electrode for wirelessly transmitting the EEG signals, and
a receiver for wirelessly receiving the EEG signals from the transmitter and transmitting the EEG signals to the sleep stage processor.

13. The system of claim 1, comprising
a second dry electrode for detecting the EEG signals of the user, positioned at or near the head of the user,
an electrode processor for receiving and processing the EEG signals from the first and second dry electrodes

14. The system of claim 13, wherein the electrode processor generates a difference between an output of the first dry electrode and an output of the second dry electrode.

15. The system of claim 14, comprising a third dry electrode for detecting the EEG signals of the user, positioned at or near the head of the user and in communication with the electrode processor, wherein the third dry electrode serves as an electrical ground.

16. The system of claim 1, comprising a memory in communication with at least one of the sleep stage processor and the pressure delivery device for storing at least one of a history of sleep stages of the user and a history of pressures at which the controlled stream of air is delivered to the user.

17. The system of claim 1, comprising
a housing containing the sleep stage processor, and
a display seated on the housing for depicting information based at least partially on the sleep stage.

18. The system of claim 17, wherein the display depicts at least one of an indicator denoting the sleep stage of the user and a respiratory event number representing an apnea-hypopnea index.

19. The system of claim 17, wherein the display depicts at least one of the EEG signals, a hypnogram corresponding to a history of sleep stages of the user, a sleep quality index representing sleep quality of the user over a period of time, and a total sleep number representing a total amount of sleep over a period of time.

20. The system of claim 1, comprising an actigraph for detecting motion signals representing movement by the user, wherein at least one of the sleep stage of the user and the pressure of the controllable stream of air is determined based, at least in part, on the motion signals.

21. The system of claim 1, wherein
the first dry electrode detects at least one of a level of muscle tone of the user, an EOG signal, and a galvanic skin response,
and the sleep stage processor determines the sleep stage based at least partially on at least one of the level of muscle tone, the EOG signal, and the galvanic skin response.

22. The system of claim 1, wherein the sleep stage processor applies a neural network when processing the EEG signals to determine the sleep stage of the user.

23. The system of claim 1, wherein the first dry electrode comprises a conductive fabric disposed in contact with skin of the user.

24. The system of claim 23, wherein the conductive fabric includes at least one of silver, copper, gold, and stainless steel.

25. The system of claim 1, wherein a portion of the first dry electrode in contact with skin of the user is flexible.
